(19) United States
(12) Patent Application Publication
(10) Pub. No.: US 2018/0343857 A1
(43) Pub. Date: Dec. 6, 2018

(54) BIOSOLUBILIZER

(71) Applicant: SOLIANCE, Pomacle (FR)

(72) Inventors: Cédric ERNENWEIN, Nouvion le Vineux (FR); Romain REYNAUD, Reims (FR); Arnaud GUILLERET, Boult sur Suippe (FR); Lucie PODEVIN, Henin-Beaumont (FR); Alexis RANNOU, Laon (FR); Frédérique LAFOSSE, Reims (FR)

(21) Appl. No.: 16/041,897

(22) Filed: Jul. 23, 2018

Related U.S. Application Data
(62) Division of application No. 14/405,935, filed on Dec. 5, 2014.

(30) Foreign Application Priority Data
Jun. 6, 2012 (FR) 1201629
May 24, 2013 (FR) PCT/FR2013/000133

(51) Int. Cl. A01N 25/30; C11B 9/00; C11D 1/66; B08B 1/00; A01N 57/20; A61K 47/26; A61Q 19/00; C11D 3/22; C11D 3/43; A61Q 5/00; A61K 8/60; A01N 31/16; A01N 37/02; A01N 65/00; A01N 65/22; A01N 65/36

(52) U.S. Cl. CPC A01N 25/30; C11B 9/00; A01N 65/36; B08B 1/001; A01N 57/20; A61K 47/26; A61Q 19/00; C11D 3/221; C11D 3/43; A61Q 5/00; A61K 8/602; A61K 2800/49; A01N 31/16; A01N 37/02; A01N 65/00; A01N 65/22; C11D 1/662

(57) ABSTRACT

Composition containing at least one substance that is not water-soluble and is solubilised in a sophorolipid, and at least water, and use thereof for cleaning hard surfaces and laundry, for killing plants, for combating insect pests, for combating fungus and mould proliferation, for combating gastropods, for combating moss, algae and lichen proliferation, for the cosmetics industry and for skin and hair care.

### CROSS-REFERENCE TO RELATED APPLICATION

This application is a division of U.S. patent application Ser. No. 14/405,935 filed on Dec. 5, 2014, which is incorporated herein by reference, which was based upon and claimed the benefit of priority from the prior PCT/FR2013/000133 filed on May 24, 2013 and French Patent Application No. 1201629 filed on Jun. 6, 2012; the entire contents of which are incorporated herein by reference.

The present invention relates to compositions containing at least one biosurfactant from the family of glycolipids, and more particularly a sophorolipid and a compound that is not water-soluble. The biosurfactant is then used as a solubilising agent for the material that is not water-soluble.

The concept of solubilisation was first defined by McBain in 1918 in the “Journal of the Chemical Society” 113, 825. According to this definition, solubilisation involves the spontaneous passage of a molecule that is not water-soluble (or partly insoluble) into a given solvent forming a thermodynamically stable solution. Here the solvent enabling the solubilisation is an aqueous solution of surfactants. The solubilisation takes place because of the formation of aggregates of surfactant molecules, known as micelles, forming non-aqueous nano-domain in water. Solubilisation can take place by incorporating insoluble molecules into the core of micelles or by their adsorption to the surface of micelles or even by their aggregation into the membrane forming the wall of the micelle.

The use of glycolipidic biosurfactants in the pharmaceutical, cosmetic and detergent fields or also the formulation of phytosanitary products is known. The preferred glycolipids are generally sophorolipids as, in addition to their effectiveness and features in the cited fields of application, they are industrially available and competitive.

Sophorolipids are formed by a saccharide part linked by a glycosidic bond to a remainder of fatty alcohols, fatty acids or fatty acid esters. The saccharide part, forming the polar part or “polar head” of the surfactant, is composed by a remainder of sophorose comprising free or acetylated hydroxyls. Sophoroses are particular sugars composed of two glucose units linked in beta 1,2. Sophorolipids with a remainder of fatty acid as a lipophilic part, also known as a “lipophilic tail”, have at least one carboxylic acid function in acid form or in separated form, in particular from alkali metal salts. Said carboxylic function can be esterified by a compound comprising at least one alcohol function, in particular methanol. For long alkyl radicals, i.e. comprising at least 10 carbon atoms, and preferably 16 to 18 carbon atoms, an intramolecular esterification with one of the free hydroxyls of the glucoses of the polar head is possible. This is referred to as a lactone form. Generally, commercially available sophorolipids are mixtures of acid or ester open forms, with lactone forms or closed forms, di-acetylated, monoacetylated or non-acetylated forms.

Sophorolipids are part of the bio-surfactant family. The term ‘bio’ refers to their method of production by biotechnology. They are in fact obtained by a process of fermentation. This process was described in particular by Gorin and collaborators in the “Canadian Journal of Chemistry”, 39, 846 in 1961. Natural bacteriological, selected or modified strains capable of metabolising saccharide sources and lipids in order to form glycolipids, and in particular sophorolipids, are for example Candida apicola, Candida bombicola, Yarrowia lipolytica, Candida bogoriensis.

In the fields of pharmaceuticals and cosmetics the biological activity of sophorolipids has been described in particular. The biological activity is generally connected to a bactericide, bacteriostatic, fungicide, virucide, antimicrobial or spermicide activity. WO 2006/069175 A2 describes for example an antifungal activity, WO 2005/089522 a spermicide and virucide activity and WO 2007/130738 an action for combating herpes. For said fields of application the lactone forms are generally isolated, in particular by complex and costly methods of chromatography. Some of these require chemical functions, in particular esterifications, and acetylation of the hydroxyls of the polar head to exert their biological activities.

In the more specific field of cosmetics, sophorolipids are used for skin or hair applications. WO 97/01343 relates for example to an application associated with the depigmentation of skin, WO 2004/108063 to the control of adipose mass and EP 0209783 to anti-dandruff activity.

Sophorolipids, in isolated, purified or chemically modified forms are therefore used as molecules with a targeted biological activity. They are therefore formulated like those with adjuvants of formulations, in particular, emulsifiers and other surfactants, in dermatological or capillary preparations, in the form of oil-in-water emulsions (O/W) or water-in-oil emulsions (W/O) or solutions.

The use of raw forms of sophorolipids, i.e. emerging directly from the fermentation process, or extracts from the fermentation medium, as a solubilising agent of compounds that are not fully soluble in water has never been described. Not fully soluble in water refers to compositions that are not soluble in water in a temperature range varying from 0 to 100° C., or are partially soluble, i.e. the solubility range of the compound in water is limited in concentration and/or in temperature and/or in the ionic force of the dilution water. Organic molecules are not completely soluble, as are non-ionic surfactants with cloud points lower than 100° C., and ionic surfactants with Krafft points or crystallisation points greater than 0° C.

In the field of detergents, the use of glycolipids, and more particularly sophorolipids, relates to surfactant properties, in particular to the activities of cleaning laundry and hard surfaces. Document EP B0499434 refers to the effectiveness of compositions containing sophorolipids in a mixture with other anionic or non-ionic surfactants for cleaning laundry. EP 1411111 A1 refers to weakly foaming compositions containing sophorolipids, in particular mixtures of lactone forms and open forms. EP 1445302 A2 relates to compositions for cleaning hard surfaces, in particular polycarbonate surfaces, containing glycolipids in a mixture with surfactants not forming lamellar phases. In all of these examples, the properties revealed are associated with the synergy effects of mixtures of surfactants containing at least sophorolipids for cleaning or even the weak production of foam of sophorolipids in solution.

The solubilisation of compounds not completely soluble by using sophorolipids combined with strengthening the cleaning action has never been described. In particular, the solubilisation of weakly polar or non-polar solvents by sophorolipids to increase the cleaning action, more particularly degreasing, of hard surfaces has never been shown.

Patent application WO 2011/051161 A1 describes compositions based on sophorolipids and solvents. The patent application relates to the formulation of a cleaning agent for hard surfaces which leaves few traces after drying, particularly on glass. The sophorolipids are used here solely with polar solvents that are soluble in water, in particular alcohols and glycol ethers. In the description of the invention and examples, the authors claim that it is necessary to add solubilising agents and/or hydrotropes, in particular sodium xylene sulfonate and/or ethanol to stabilise the solutions.

### DESCRIPTION OF THE INVENTION

The present invention relates to the solubilisation ability of sophorolipids and the use of sophorolipids as a solubilisation agent in cosmetic preparations, hair and skincare products, preparations for cleaning laundry and hard surfaces, as well as in preparations for treating plants.

A particular feature of the present invention relates to compositions for cleaning hard surfaces containing at least one sophorolipid, a non-aqueous, non-polar solvent and water. These particular compositions according to the invention exhibit remarkable degreasing properties because of the combined action of sophorolipids and solvents. Moreover, they do not necessarily contain volatile and inflammable alcohols, in particular no ethanol and no glycol ethers, which represents undeniable technical progress.

One of the advantages of solubilising agents is that they make it possible to obtain perfectly clear solutions, even in the presence of insoluble molecules. In a general manner the solution is composed mostly of water and said insoluble substance would result in a turbid solution or an additional distinct phase without the addition of solubilising agent.

The clarity of the solution in the presence of the solubilising agent and said insoluble substance can be determined in different ways, in particular by means of a turbidity measuring instrument equipped with a 860 nm light source or spectrometer for measuring absorbance by means of a 600 nm light source. In both cases, the clarity is ensured if the absorbance is between 0 and 0.1, preferably between 0 and 0.05 or if the NTU value (Nephelometric Turbidity Unit) is less than 20, preferably less than 10. Another method consists of placing 20 g of the liquid to be evaluated into a 30 ml glass flask, placing a white sheet with 2 cm square black squares behind the flask (thus forming a black and white checked pattern). The clarity is assessed visually by placing a flask containing filtered water (0.45 μm) next to the flask containing the solution to be evaluated. The solution is clear if the cleanness of the black and white squares is the same as with the filtered water.

The preferred method for obtaining a clear solution consists of first stage of adding the insoluble substance to a concentrated aqueous solution containing 100 to 10% by weight, preferably 100 to 50% solubilising agent, by agitating the mixture at a temperature of between 10 and 100° C., preferably between 20 and 60° C., more preferably at ambient temperature. The mixture obtained is agitated continuously, for example by means of a motor and agitating blade, for example also by means of a magnetic bar and a rotary magnet placed underneath the flask, for example also by means of ultrasound, a rotor-stator type agitator, a colloidal mill or by manual agitation. The speed of agitation can vary from 1 to 50000 rotations per minute, preferably 1 to 100 rotations per minute. The duration of the agitation is generally between 0.05 and 300 minutes, preferably between 1 and 10 minutes. After this stage of thorough mixing between the solubilising agent and the substance to be solubilised, the other constituents are then added to this pre-mixture during continuous agitation. In a preferred manner, the amount of water necessary or the remaining portion of water is added last to obtain a clear solution as defined above.

The mass ratio between the solubilising agent and the solubilised material varies from 1 to 9000 and preferably from 1.6 to 90, and water composes 1 to 99.89% of the total mass of said composition.

The solubilisation performance can be expressed either in an empirical manner or by using predictive tools. Of all of these tools the Hansen parameters are the ones most commonly used. The Hansen theory continues the principle of the Hildebrand global solubility parameter, which is defined as the square root of the cohesive energy density by unit of volume. The cohesive energy being connected to the vaporisation enthalpy according to the principles of thermodynamics has a measurable size. Nevertheless, the parameter of global solubility seems insufficient for describing the phenomena of solubility, in particular the interactions between molecules. The Hansen parameters decompose the Hildebrand global solubility parameter into a dispersive component or δd, polar or δρ and hydrogen bonding force or δh (equation 1). They are described in the book “Hansen Solubility Parameters: A user's handbook” by Charles M. Hansen published by CRC Press, and can be calculated by means of software according to the group contribution method, in particular according to the method of Van Kervelen (“Properties of Polymers” published by Elsevier, in 1990. The MPa^{1/2} is the most widespread SI unit, but there is an earlier unit, Cal^{1/2} cm^{-3/2}, which is still used sometimes. To convert this earlier unit into an SI unit, it is necessary to multiply the corresponding parameter of solubility by 2.0455.

δ²=δ²_{d}+δ²_{P}+δ²ₕ Equation 1

The non-aqueous, non-polar solvents used in the compositions of the present invention include solvents selected from those with at least one area of non-solubility in water and characterised by a Hansen polar parameter or δρ of less than 5.5 Mpa^{1/2}, preferably less than 5.1 Mpa^{1/2}, preferably even lower than 4.1 Mpa^{1/2}.

Another particular feature of the invention relates to the solubilisation of insoluble active ingredients, perfumes, essential oils, colorants, pigments insoluble in water in preparations containing at least one sophorolipid and water. It has been found that sophorolipids can be used as solubilising agents, in particular for the cosmetics and pharmaceutical sectors. In these sectors the most commonly used solubilising agents contain at least one polyoxyethylenated derivative, very often a polyethoxylated castor oil with 40 ethylene oxide units, such as Cremophor CO40 of BASF. The substitutes of these polyethoxylated solubilising agents, justified by the search for ingredients not using ethylene oxide or propylene oxide, do not generally have an equivalent level of effectiveness. A person skilled in the art often has access to mixtures of surfactants and solvents for increasing the solubilisation ability for a range of molecules to be solubilised. The sophorolipids have remarkable solubilisation abilities in the aqueous phase, without it being necessary to add other surfactants or solvents. Furthermore, they do not contain a unit of ethylene oxide or propylene oxide, are produced solely from renewable materials and are made by a less efficient fermentation process than the processes of standard chemistry. In this way the sophorolipids make it possible to deal with this lack of solubilising agent of vegetable origin and plus particularly of biosolubilising agent.

Documents WO2004/108063, FR2779057, U.S. Pat. No. 6,057,302, U.S. Pat. No. 5,756,471, EP0209783, WO2011/051161, U.S. Pat. No. 6,262,038 and JP2009275145 do not describe a clear composition.
The sophorolipids of the present invention have the following general structures:

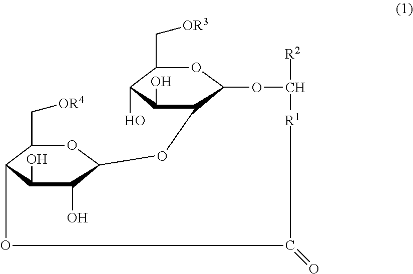

R1 and R1′ are independently of one another saturated hydrocarbon chains or have one or more non-hydroxylated unsaturations or have one or more hydroxyl, linear or ramified groups with 1 to 21 carbon atoms, in particular derived from an alkyl radical.

R2 and R2′ are independently of one another a hydrogen atom or a saturated alkyl radical or have one or more non-hydroxylated unsaturations or have one or more hydroxyl, linear or ramified groups with 1 to 9 carbon atoms.

R3, R4, R3′ and R4′ are independently of one another a hydrogen atom or an acetyl group.

R5 is an OCH₃ or OH or O⁻M⁺ group where M+ is a metal ion or an organic cation, in particular ammonium salts such as dimethylammonium, trimethylammonium, isopropylammonium, monoethanolammonium, diethanolammonium, triethanolammonium, disphosphonium or O(CH₂)ₘCH₃ with m being between 1 and 11.

Both compounds (1) and (2) or one or the other compound form the sophorolipid of compositions of the invention.

Industrially available sophorolipids contain impurities in addition to compounds (1) and/or (2). The impurities can be fatty alcohols, fatty acid esters, triglycerides or oils, sugars, in particular glucoses, sophoroses, organic acids in their acid or dissociated forms, in particular fatty acids, acetic acid.

The molecules that can be solubilised in aqueous phase by the sophorolipids in preparations according to the invention can be active, that is molecules with a targeted, biological or chemical action.

The sophorolipids are therefore indispensable technological adjuvants which enable in particular the formation of solutions, lotions, aqueous gels and microemulsions containing one or more solubilised active ingredients.

The following is a list of active ingredients, but the list should not be considered to be restrictive:
vitamins, such as vitamin A, E or C,
anti-inflammatories, such as plant extracts, alpha-bisabolol, panthenol, alpha-tocopherol,
anti-scalding agents, such as allantoin,
anti-ageing agents, such as retinol,
agents for pigmenting the skin and/or agents for depigmenting the skin, such as kojic acid, coumaric acid, arbutin,
slimming agents, such as caffeine, phytosterols, phloridizin, extracts of quinoa,
anti-perspirant agents, such as aluminium salts, zinc or zirconium salts, diethylene triamine pentaacetic acid,
anti-dandruff agents, such as zinc or aluminium pyrithione, salicylic acid, pyridone salts and derivatives of piperazine,
organic UV-A and/or UV-B sun filters for protecting the skin or hair from the effects of the sun and UV rays, such as compounds authorised by European directive No. 76/768/CEE, its appendices and final modifications, and in particular derivatives of benzophenone, cinnamic acid esters, salicylic acid esters, 3-benzylidene camphor,
antioxidants, such as ascorbic acid and derivatives thereof, citric acid and derivatives thereof, glutamic acid, glutamates and derivatives thereof, lactic acid and the derivatives thereof, tartric acid and the derivatives thereof, bioflavonoids, buthylhydroxy hydroxyanisol, carotene and the derivatives thereof, sulphites such as sodium bisulphites, chlorobutanol,
preservatives such as parabens, phenoxyethanol, formaldehydes, pantane diol, sorbic acid,
insect repelling agents such as acetamiprid, etofenprox, permethrin, cypermethrin, N,N-diethyl-m-toluamide, butyl acetyl-aminopropionate,
active pharmaceutical ingredients, such as disinfectants, such as derivatives of chlorexidrine, benzoic acid, anti-inflammatories, such as tincture of arnica, eucalyptol, menthol, dimethoxy-1,2-benzene, anti-acne agents such as derivatives of tretinoid, azelaic acid, salicylic acid, anti-scalding agents, such as derivatives of triethanolamine, antifungals, such as derivatives of pyridone, such as cyclopiroxolamine, derivatives of imidazole, such as clotrimazole, folic acid, riboflavin,
natural or synthetic pigments that are not soluble in water,
natural or synthetic aromas that are not soluble in water,
active phytosanitary ingredients, such as herbicides, fungicides, insecticides such as those described in “The Pesticide Manual” (9th edition, C. R. Worlkling and R. J Hance, editors, published by the British Crop Protection Council) in their forms that are not soluble in water.

Another use of the present invention relates to the formulation of compositions containing at least one perfumed compound or essential oil. The fragrant compounds that are not water-soluble are put in contact with a solution containing at least one sophorolipid which enables their complete solubilisation in the solution containing water. The following is a list of perfumes and essential oils, but the list should not be considered to be restrictive:
synthetic or natural perfumes, such as benzyl acetate, linalyl acetate, benzyl acetate, terpenyl acetate, vetyveryl acetate, amyl acetate, bornyl acetate, cedryl acetate, geranyl acetate, phenylethyl acetate, paracresyl acetate, styrallyl acetate, amyl butyrates, linalyl benzoate, citral, citronellal, lilial, eugenol, geraniol, citronelol, linalool and other terpenic derivatives, anisic alcool, cinnamic alcohol, styrallic alcohol, aldehydes such as, octylic, nonylic, decylic, undecylenic, lauric, myristic, cetylic, stearic, benzoic, anisic aldehydes, synthetic camphor, limonene,
essences of sage, camomile, carnation, vetiver, lavender,
essential oils, such as the essential oils of lavender, thyme, savory, sage, mint, cumin, caraway, star anise, fennel, dill, eucalyptus, cajeput, niaouli, cloves, pine, cedar, cypress, juniper, lemon, orange, bergamot, cinnamon, laurel, camomile.

Another use of the present invention relates to the formulation of a product for cleaning hard surfaces and fabrics.

In addition to the presence of active ingredients that are not water-soluble, perfumed compounds or essential oils that are not water-soluble as described above, the compositions can contain non-polar solvents that are not water-soluble.

The sophorolipids are added to the cleaning composition both for their good surfactant properties and also for their solubilisation ability. The solvents are added in order to increase the cleaning performance, in particular the degreasing. The formulation of solutions, lotions, aqueous gels, microemulsions containing at least one non polar, non-aqueous solvent being preferable to other formulations, in particular emulsions of oil in water, as they are more stable over time.

The non-polar solvents are those for which the Hansen polar solubility parameter (denoted dp) is less than 5.5 MPa^{1/2}, preferably less than 5.1 MPa^{1/2}, and more preferably less than 4.1 MPa^{1/2}.

The following are listed in this category without this being a restrictive list:
alkanes, in particular heptane, hexane, pentane, octane, decane, dodecane, hexadecane and other alkanes with Hansen polar solubility parameters of 0,
de-aromatised or partly de-aromatised paraffinic oils, such as light distillates of petrol, white spirit, linear hydrocarbons and/or ramified hydrocarbons with C8 to C18, cyclic hydrocarbons, said solvents having a Hansen polar solubility parameter of 0 or less than 1,
aromatic hydrocarbons, in particular light naphta oils (dp=0.7), naphtalene (dp=2.0), benzene (dp=0), toluene (dp=1.4), ethyl-benzene (dp=0.6), oxylene (dp=1), toluene (dp=1.4), o-n-butyltoluene (dp=0.1),
chlorinated derivatives, in particular chloroform (dp=3.1), 1,1,2 trichloroethylene (dp=3.1),
vegetable or animal oils, in particular lard, suet, groundnut oil, butter oil, cottonseed oil, flax oil, olive oil, palm oil, grapeseed oil, fish oil, soya oil, castor oil, rapeseed oil, copra oil, coconut oil, sesame oil, pine oil, vegetable oils having generally Hansen polar solubility parameters of between 3 and 4,
medium chain triglycerides, in particular triglyceride C8/C10 or MCT (dp=3.6),
esters of vegetable oils, in particular methyl rape ester, methyl sunflower ester, methyl oleate, isopropyl myristate, amyl laurate, isostearyl stearate, esters of vegetable oils with Hansen polar solubility parameters of generally less than 2,
fatty acids from vegetable oils, in particular oleic acid (dp=3.1), octanoic acid (dp=3.3),
acetates, in particular butyl acetate (dp=3.7), isobutyl acetate (dp=3.7), amyl acetate (dp=3.3), isopropyl acetate (dp=3.3),
terpenes, in particular limonene (dp=1.8), p-cymene (dp=0.6), alpha-pinene (dp=4.3), farnesene, farnesol (dp=3.8),
alcohols, in particular oleic alcohol (dp=2.6), 2-ethyl-hexanol (dp=3.3), octanol (dp=3.3), nonanol, isodecanol, decanol (dp=2.7)l, dodedanol, cyclohexanol (dp=4.1), amylic alcohols (dp=4.7), tridecanol (dp=3.1), hexyldecanol, butyl-octanol,
carbonates, in particular dimethyl carbonate (dp=3.9), diethyl carbonate (dp=3.1), di-n-propyl carbonate (4.1), 1,2-dodecane carbonate (dp=2.6),
organic acid esters, in particular diethyl succinate (dp=4.1), dibutyl succinate (dp=2.9), diamyl succinate (dp=3.−7), diisooctyl succinate (dp=2.7), dioctyl succinate (dp=2.7), didecyl succinate (dp=2.3), dibasic esters (DBE esters of the company Invista) (dp from 4.3 to 5.1), DBE-IB of Invista (dp=2.6), Rhodiasolv RPDE, IRIS DIB of the company Rhodia,
amines, in particular trimethyl amine (dp=3.4), 3-methoxy-propylamine (dp=3.9), butylamine (dp=4.5) cyclohexylamine (dp=3.1), diethylamine (dp=2.25), diallylamine (dp=4.5), octylamine, decylamine, oleylamine,
ethers, in particular methylal (dp=1.8), triethylene glycol monooleyl ether (dp=3.1), methyl-t-butyl ether (dp=3.5).

Compositions containing at least one sophorolipid and a polar solvent soluble in water, in particular methanol, ethanol, isopropanol, ethylene glycol diethyl ether, ethylene glycol dibutyl ether, ethylene glycol butyl ethyl ether, ethylene glycol monoisopropyl ether, propylene glycol monobutyl ether, propylene glycol monopropyl ether are excluded from the present invention. In particular, the solvents described in document WO 2011/051161 A1 are excluded from the present invention.

The subject matter of the present invention is also a composition comprising by weight 0.1 to 90%, preferably 0.1 to 35%, more preferably 1 to 15% sophorolipids and 0.01 to 65%, preferably 0.1 to 35%, and more preferably 0.1 to 5%, a material or mixture of materials that are not completely water-soluble, the remainder being formed by an aqueous solution. Said aqueous solution composing the remainder of the composition according to the invention contains at least water and possibly adjuvants of formulations that are soluble in water, in particular non-ionic, anionic, cationic surfactants, salts, pH adjusters, hydrating agents, chelates, metal ions, polymers, dispersing agents, colorants, preservatives and hydrotropes.

The following is a list of non-ionic surfactants, but the list should not be considered to be restrictive:

alkyl polyglycosides, in particular C8/C10 polyglucosides, C8/C10 wheat bran glycoside known by the commercial name Appyclean 6781 of the company Wheatoleo, C12/C14 polyglucosides, as well as polyethoxylated fatty alcohols in particular alcohol C12/C15 with 7 ethoxylated units, C9/C11 with 4 ethoxylated units, C9/C18 with 5 ethoxylated units, as well as ethoxylated fatty acids such as ethoxylated oleic acids, as well as ethoxylated sorbitan esters such as sorbitan laurate with 20 ethoxylated units, as well as ethoxylated triglycerides such as lard, suet, groundnut oil, butter oil, cottonseed oil, flax oil, olive oil, palm oil, grapeseed oil, fish oil, soya oil, castor oil, rape oil, coprah oil, coconut oil polyethoxylated, as well as alkylpolyglucosamides, as well as glucamides, as well as amine oxides such as alkyl oxides C10/C18 dimethylamines, alkoxy oxides C8/C22 ethyldihydroxyethylamines, as well as esters of ethoxylated polyglycerol such as glycereth 17 cocoate, glycereth 7 caprylate caprate, glycereth 20 stearate, as well as ethoxylated amines, as well as ethoxylated fatty amides, as well as copolymers of propylene oxide and ethylene oxides.

The following is a list of anionic surfactants, but the list should not be considered to be restrictive:

alkylbenzene sulfonates, preferably linear, as n alkyl in 010/012 sodium benzene sulfonate, as well as paraffin sulfonates with the formula R—CH(SO3M)-R′, where R and R′ are remains of paraffin and M a cation of alkali metal, as well as alkyl sulfonate esters of the formula R—CH(SO3M)-COOR′, where R is an alkyl radical of C8 to C20 and R′ an alkyl radical of C1 to C6, M a cation of alkali metal, as well as alkylsulfates with the formula ROSO3M, where R is an alkyl radical or hydroxyalkyl of C8 to C24 and M is a cation of alkali metal, as well as alkylether sulfates of the formula R—O—(CH2-CH20)n-SO3M, where R and M are defined as above and n is the number of ethylene oxide units and is generally between 0.5 and 6, preferably between 2 and 3, as well as sulfosuccinates of the formula R—O—CO—SH(SO3M)-CH2-COOH, where R and M are defined as above, preferably R is between 12 and 14, as well as alkylamide sulphates of the formula RCONHR′ OSO3M, where R represents an alkyl radical of C2 to C22, R′ an alkyl radical of C2 to C3 and M a cation of alkali metal, as well as the polyethoxylated derivatives thereof comprising 0.5 to 60 ethoxylated units, as well as saturated or unsaturated fatty acid soaps with C8 to C16, as well as alkylglycerol sulfonates, as well as N-acyl-N-alkyl taurates, alkyl phosphates, as well as polyoxyethylene carboxylates, as well as alkylisethionates, as well as alkyl succinamates, as well as N-acyl-sarcosinates, as well as alkylglycoside sulfates.

The following is a list of cationic surfactants, but the list should not be considered to be restrictive:

alkyltrimethylammonium halides, as well as dialkyldimethylammonium halides, as well as quaternary dialkylimidazolines, as well as quaternary dialkylamidoamines, as well as esters of dialkylamidazoline, as well as chlorides of dialkyesters of quarternary dihydroxypropyl ammonium, as well as methylsulfates of dialkylester of quarternary triethanolammonium.

The following is a list of amphoteric surfactants, but the list should not be considered to be restrictive:

alkyldimethyl betaines, as well as alkyl amidopropyldimethyl betaines, as well as alkyl trimethyl sulfobetaines, as well as condensation products of fatty acids and protein hydrolysates.

The following is a list of ingredients that make it possible to adjust the pH of the solution, the following are listed without being restrictive to obtain an alkali pH, soda, potash, ammonia, amino monoethanol, triethanolamine, sodium tripolyphosphate, sodium carbonate/sodium bicarbonate, silicates, in particular sodium metasilicate, sodium gluconate, sodium hydrogenophosphate. To obtain acid pH the following are listed for example sulphuric acid, hydrochloric acid, phosphoric acid, lactic acid, citric acid, glycolic acid, sulfamic acid, acetic acid, formic acid and oxalic acid.

The following are listed as complexing agents this being a restrictive list: EDTA, NTA, sodium citrate, IDS (iminodisuccinate such as baypure CX100 of the company Lanxess), MGDA (methylglycine-diacetic acid such as Trilon M of BASF), DTPA (diethylene triamine pentaacetic acid such as trilon C of BASF), HEDTA (hydroxyethyl ethylenediamine triacetic acid such as Trilon D of BASF), HEIDA (N-2-hydroxyethyl iminodiacetic acid).

As the salts sodium chloride, potassium chloride are given without any restriction thereto.

As the hydrating agents glycerol, polysaccharides such as those of hyaluronic acid are given without any restriction thereto.

The following are given as thickening and viscosity increasing agents without any restriction thereto: natural polymers, such as guar gums, xanthane gums, agar-agar, carrageenans, alginates, pectins, native or modified starches, derivatives of cellulose, such as hydroxy-ethyl-cellulose, gelatines of animal origin, as well as synthetic polymers, such as acrylic polymers, vinyl polymers, such as polyvinyl alcohols and also clays.

The following are given as hydrotropes without any restriction thereto: sodium sulphonate xylenes, p-toluene sodium sulfonate, sodium sulfonate cumene, 2-ethyl-hexyl sodium sulfonate, alkyl polyglucosides with the formula R—O-(G)n, where R is a linear or ramified alkyl group comprising 4 to 8 carbon atoms, G is a remainder of glucose, n represents the degrees of holigomerisation and is a number between 1 and 3, amyl-xylsodise, mono-alkyl isosorbides, urea, sodium sulfate butyl-monoglycol.

### Use

The compositions according to the invention are particularly effective for cleaning hard surfaces. They are therefore useful in particular for producing multi-purpose household products, degreasing sprays for kitchen surfaces, cleaning agents for the oven and hotplates, products for washing up by hand or by dishwasher, products for cleaning floors by hand or by machine, products for the bathroom, and in particular sprays for washing the bath and washbasins, gels for cleaning the toilet, sprays for cleaning glass and mirrors, washcloths for cleaning surfaces.

Compositions according to the invention can also be used for the formulation of products for textiles, in particular liquid detergents, gels or liquid stain removers, softeners, products for carpet, products for the interior of vehicles.

For cleaning the compositions according to the invention are used in domestic households but also in institutions, in particular the catering sector and hotel business, cleaning floors and furniture in offices and businesses, but also in industry, in particular for cleaning in the agro-industrial business, cosmetics, inks, lubricants, metal treatments, degreasing metal or non-metal parts, cleaning vehicles and planes.

For cleaning hard surfaces and in particular for degreasing, the composition according to the invention and containing at least one sophorolipid and a non-polar solvent that is not water-soluble is adjusted to a pH of between 3 and 12, preferably between 4.5 and 9.5 and more preferably 5 to 7.5.

The pH value of the sophorolipids is generally acidic, generally with a pH value of between 3 and 5 at a concentration of 5 to 55% in water. To lower this pH value it is necessary to add an acid to the solution containing the sophorolipids, preferably an acid of average or low strength such as citric acid, lactic acid, glycolic acid, propanoic acid, acetic acid, butyric acid, formic acid, valeric acid, benzoic acid and salicylic acid.

To increase this pH value it is necessary to add a base to the solution containing sophorolipids, preferably a weak or average strength base, such as monoethanol amine, diethanol amine, triethanol amine, triethyl amine, diethyliso propylamine, ammonia, trisodium citrate, sodium glutamate, polyphosphates, tetrapotassium pyrophosphate (or TKPP), sodium bicarbonate, phosphates such as sodium or potassium hydrogen phosphate, silicates such as sodium metasilicate.

In some cases, in particular for industrial cleaning it is necessary to increase the pH of the solutions significantly, generally to pH values greater than 10. In this case the use of a strong base is prescribed such as soda, potassium hydroxide, calcium hydroxides.

For domestic cleaning, degreasing sprays are preferably adjusted to a pH of between 5.5 and 7, which makes it possible to propose effective products that are compatible with the skin and mucosa.

For their solubilisation properties of active ingredients, the essential oils and perfumes in the compositions according to the invention are also used for making personal care products. In particular, for making shower gels, hand soaps, face creams, shampoos, conditioners, hair products, bath products, cleansing lotions, skin treatment products, in particular for treating acne, lotions for improving the complexion or brightness of the skin, anti-ageing lotions, lotions for protecting the skin from external exposure, in particular from the sun, lotions for cleaning the oral cavity, lotions for cleaning genital areas.

The combined use of sophorolipids with at least one non-polar, non-aqueous solvent according to the invention also makes it possible to improve the wetting of apolar surfaces, improve the quality of spray jets and improve foliar penetration, which makes them effective for treating plants.

The compositions according to the invention are also used in the field of phytosanitary treatments, in particular for the treatment of plants or the destruction thereof, for combating insect pests, for combating fungus and mould proliferation, for combating gasteropods, for combating moss, algae and lichen proliferation.

In this field, the compositions according to the invention can contain one or more active ingredients. The latter are referred to as “ready-mix” or ready-to-use formulas.

The compositions can be without active ingredients, but have additional functions, in particular wetting, penetrating, anti-drift, foaming, anti-foaming, complexing ions in particular calcium and magnesium, adjustment of pH, colorants. The latter are referred to as “tank-mix” formulas or extemporaneous adjuvants, the product being added before application in addition to another product containing an active ingredient.

### EXAMPLES

In all of the following examples the sophorolipids used are produced by company WHEATOLEO under the commercial name “SOPHOCLEAN” and/or by the company SOLIANCE under the commercial name “SOPHOLIANCE S”. They are produced by a method of fermentation using Candida bombicola, which enables the metabolising of glucose and methyl rape esters into sophorolipids. The commercial products contain at least 70% by mass sophorolipids relative to the dry mass, the remainder being formed mainly by sugars and fatty acids. The sophorolipids of the two commercial products are defined by structures (1) and (2) described above.

The main form is a lactone form, form (1) C18:1, i.e. the hydrophobic part comprises 18 carbons and one unsaturation and has been subjected to intramolecular esterification. According to (1), R2 is a methyl group, R1 is a linear alkyl with 15 carbon atoms and one unsaturation. R3 and R4 are acetyl groups. Said form represents from 15 to 40% of the total dry mass.

The second form, representing 10 to 40% of the dry mass is an open form C18:1, that is the apolar part is formed by the same remainder of fatty acids as above, but has not been subjected to esterification. According to (2), R2′ is a methyl group, R1′ is a linear alkyl with 15 carbon atoms and one unsaturation, R3′ and R4′ are acetyl groups and R5 is an OCH3 group.

The third form representing 5 to 25% of the dry mass is an open form C18:1. According to (2), R2′ is a methyl group, R1′ is a linear alkyl with 15 carbon atoms and one unsaturation, R3′ and R4′ are acetyl groups and R5 is an OH group.

The fourth form representing 5 to 25% of the dry mass is an open form C18:2. According to (2), R2′ is a hydrogen, R1′ is a linear alkyl with 16 carbon atoms and two unsaturations, R3′ and R4′ are acetyl groups and R5 is an OCH3 group.

The fifth form representing 0 to 10% of the dry mass is a lactone form C18:2. According to (1), R2 is a methyl group, R1 is a linear alkyl with 15 carbon atoms and two unsaturations and R3 and R4 are acetyl groups.

The main impurities are oleic acid which represents less than 20% of the dry mass and linoleic acid which represents less than 3% of the dry mass.

In the following the terms sophorolipids, SOPHOLIANCE S and SOPHOCLEAN define all of the commercial products defined above.

### 1. Demonstration of the Power of the Solubilising Agent

### 1.1 Solubilisation of Colorants that are not Water-Soluble

The trials were performed using two colorants that are not water-soluble, Rouge de Soudan 7B (MM=379.46 g/mol) produced by Acros Chemical, and Jaune au gras W1201 (MM=273.28 g/mol) produced by the company LCW. These two molecules are synthetic colorants comprising aromatic cores and nitrogen functions.

The principle of the method is to put an aqueous solution of sophorolipids with a given concentration (here from 0.1 to 100 g/l) in contact with an excess of pigment in solid form (one to two spatula tips). The solution is agitated for 3 hours by means of a magnetic bar and an agitating plate at laboratory temperature (23+/−3° C.), then filtered by means of a single-use filter of cellulose acetate with a cut-off point of 0.45 μm. The filtrate composed from the solution of sophorolipids and solubilised pigment is recovered and the amount of pigment measured in mg/1 by means of a spectrometer that absorbs in the UV and visible range (Hitachi U29000). The action is performed at the maximum absorption wavelength of the given pigment, the base line and the reference solution being a solution of sophorolipids not containing the pigment. The device is calibrated in the same conditions by solutions of pigments in ethanol.

The following table shows the results obtained expressed in mg/1 of pigment solubilised by g/L of dry matter of sophorolipids:

| | Jaune au gras | Jaune au gras | Jaune au gras |
|---|---|---|---|
| Concentration | solubilised at | solubilised at | solubilised at |
| of SOPHOLIANCE S | pH = 6 | pH = 8 | pH = 10 |
| 0.1 g/l | 0.16 mg/l | 0.36 mg/l | |
| 1 g/l | 0.33 mg/l | 0.59 mg/l | 1.14 mg/l |
| 10 g/l | 3.54 mg/l | 2.01 mg/l | 2.26 mg/l |
| 100 g/l | 95.24 mg/l | 75.69 mg/l | 66.77 mg/l |
| | Rouge | Rouge | Rouge |
| | Soudan 7B | Soudan 7B | Soudan 7B |
| Concentration | solubilised at | solubilised at | solubilised at |
| of SOPHOLIANCE S | pH = 6 | pH = 8 | pH = 10 |
| 0.1 g/l | 1.91 mg/l | | |
| 1 g/l | 7.30 mg/l | 1.85 mg/l | 3.2 mg/l |
| 10 g/l | 51.66 mg/l | 9.88 mg/l | 7.83 mg/l |
| 100 g/l | 632.05 mg/l | 316.11 mg/l | 256.98 mg/l |

### 1.2 Solubilisation of Perfumes and Essential Oils

This example seeks to demonstrate the solubilisation ability of sophorolipids in relation to perfumed materials, in particular synthetic perfumes and essential oils. The method consists of finding the minimum mass of sophorolipids necessary for solubilising one gram of compound that is not water-soluble. In practice, it consists of finding the minimum amount of sophorolipids necessary for solubilising 200 μl perfumes and/or oils supplemented to 5 g with water. The minimum mass is found by dichotomy, by observing solutions at rest at laboratory temperature (23+/−2° C.). The aim is to obtain perfectly clear solutions in which the substance is completely solubilised.

The following table shows the results obtained expressed in g of dry matter of sophorolipids necessary for solubilising 1 g of material that is not water-soluble.

| Essential | | |
|---|---|---|
| oil of | SOPHOLIANCE S at pH = 6 | SOPHOLIANCE S at pH = 8 |
| Grapefruit | 3.11 | 15.85 |
| Thyme | 9.02 | 3.24 |
| Bergamot | 7.46 | 6.35 |
| Rosemary | 2.47 | 7.49 |
| Mint | 19.37 | 5.36 |
| Lavender | 8.05 | 5.38 |

### 1.3 Solubilisation of Active Cosmetic Ingredients

The method of example 1.2 is reproduced using insoluble active cosmetic ingredients.

| | SOPHOLIANCE | SOPHOLIANCE |
|---|---|---|
| Active material | S at pH = 6 | S at pH = 8 |
| Eugenol | 22.22 | 4.78 |
| α-tocopherol | 6.52 | 4.47 |
| Menthyl lactate | 17.72 | |

### 1.4 Comparative Examples

The solubilisation ability of the sophorolipids is compared with that of Cremophor CO40 of the company BASF. The name INCI of the latter is PEG 40 hydrogenated castor oil, which is considered to be a reference material in the field of solubilising agents.

For the essential oil thyme, the solubilisation ability of Cremophor CO40 is measured to be equal to 13.15 g dry matter for solubilising 1 g oil, compared with the result 3.24 g for 1 g oil in the case of sophorolipids at a pH equal to 8 and 9.02 g for 1 g at pH=6.

For the essential oil mint, the result for Cremophor CO40 is 16.14 g for solubilising 1 g oil, which is necessary to compare with 5.36 g for 1 g if sophorolipids are used at pH=8 and at 19.37 g for 1 g at pH=6.

For α-tocopherol, one of the forms of vitamin E, the solubilisation ability of Cremophor CO40 is 6 g for 0.2 g active ingredient (data from the technical documentation of BASF). That of sophorolipids at pH=8 is 0.9 g for 0.2 g active ingredient and 1.3 g for 0.2 g active ingredient at pH=6.

### 2. Examples of Compositions According to the Invention

The following compositions show clear and stable solutions over several months at 4° C., 20° C. and 35° C. The percentages are expressed as mass of product per 100 g composition.

### Composition 2.1: Solubilisation of a Methyl Rape Ester (Insoluble)

| | |
|---|---|
| SOPHOCLEAN (DM = 52%) | 90.4% |
| Methyl rape ester (Radia 7961, Oleon) | 7% |
| Water | 2.6% |

DM: dry matter

Taking into account the amount of water present in SOPHOCLEAN, the percentage of water in the composition is 46% by weight.

Said composition is thinnable, i.e. with the addition of additional water the solution remains clear and stable.

### Composition 2.2: Solubilisation of RhodiaSolv IRIS (Solubility in Water=2.5% by Weight According to the Technical Documentation of the Company Rhodia)

| | |
|---|---|
| SOPHOCLEAN (DM = 52%) | 67.3% |
| Rhodiasolv IRIS (Rhodia) | 13% |
| Water | 19.7% |

Taking into account the amount of water present in SOPHOCLEAN, the percentage of water present in the composition is 52% by weight.

Said composition is thinnable in water.

Composition 2.3: Anti-graffiti formulation.

| | |
|---|---|
| SOPHOCLEAN (DM = 54.6%) | 50% |
| Rhodiasolv IRIS (Rhodia) | 31% |
| Serdet DSK 40 (sodium 2-ethylhexyl sulfate, | 17% |
| 40%, Elementis) | |
| Glycolic acid (67%, Across Organic) | 2% |

Taking into account the amount of water present in Sophoclean and Serdet DSK40, the percentage of water present in the composition is 32.9% by weight.

Cleaning trials performed on paint on porous materials (breeze blocks) show the effectiveness of the preparation as an anti-graffiti agent used in pure or diluted form.

### Composition 2.4: Solubilisation of Dertol 90 (Terpineol, Insoluble)

| | |
|---|---|
| SOPHOCLEAN (DM = 52%) | 88.5% |
| DERTOL 90 (DRT) | 6.9% |
| Water | 3.5% |

Taking into account the amount of water present in SOPHOCLEAN, the percentage of water present in the composition is 46% by weight.

Said composition is thinnable.

### Composition 2.5: Solubilisation of DI-Ethyl Succinate

| | |
|---|---|
| SOPHOCLEAN (DM = 52%) | 84.6% |
| D.E.S (Bioamber) | 12% |
| Water | 3.4% |

Taking into account the amount of water present in SOPHOCLEAN, the percentage of water present in the composition is 44% by weight.

Said composition is thinnable.

### Composition 2.6: Formulation of a Concentrated Degreasing Agent

| | |
|---|---|
| SOPHOCLEAN (DM = 52%) | 53.3% |
| D.E.S (Bioamber) | 30% |
| Appyclean 6505 (DM = 60%, amyl xyloside, | 16.7% |
| Wheatoleo) | |

Taking into account the amount of water present in SOPHOCLEAN and Appyclean 6505, the percentage of water present in the composition is 32.3% by weight.

### Composition 2.7: Solubilisation of Rhodiasolv RPDE (Solubility=5.7% by Weight According to the Technical Documentation of the Company Rhodia)

| | |
|---|---|
| SOPHOCLEAN (DM = 52%) | 76.9% |
| Rhodiasolv RPDE (Rhodia) | 20% |
| Water | 3.1% |

Taking into account the amount of water present in SOPHOCLEAN, the percentage of water present in the composition is 40% by weight.

### Composition 2.8: Solubilisation of Rhodiasolv RPDE (Solubility=5.7% by Weight According to the Technical Documentation of the Company Rhodia)

| | |
|---|---|
| SOPHOCLEAN (DM = 52%) | 44.2% |
| Rhodiasolv RPDE (Rhodia) | 43% |
| Appyclean 6505 (DM = 60%, amyl xyloside, | 12.8% |
| Wheatoleo) | |

Taking into account the amount of water present in SOPHOCLEAN and Appyclean 6505, the percentage of water present in the composition is 26.3% by weight.

### Comparative Example: Lotion for the Treatment of Acne: Solubilisation of Salicylic Acid

| | Composition | Comparative |
|---|---|---|
| | 2.9 | composition 2 |
| SOPHOLIANCE S (DM = 55%) | 8.0% | 0% |
| Salicylic acid | 0.5% | 0.5% |
| Appyclean 6505 (DM = 60%, amyl | 8.0% | 8.0% |
| xyloside, Wheatoleo) | | |
| Glycerol | 5.0% | 5.0% |
| Citric acid (20%) | quantity | quantity |
| | sufficient for | sufficient for |
| | pH = 4.5 | pH-4.5 |
| Water | quantity | quantity |
| | sufficient for | sufficient for |
| | 100% | 100% |
| Aspect after 24 h in | Clear solution | Cloudy solution |
| refrigerator (4° C.) | | with crystals |

### Composition 2.10: Formulation of a Product for Combating Roof Moss: Solubilisation of Perlagonic Acid (Nonanoic Acid).

| | Composition |
|---|---|
| | 2.10 |
| SOPHOCLEAN (DM = 52%) | 19.2% |
| Nonanoic acid (Aldrich) | 30.0% |
| Appyclean 6505 (DM = 60%, amyl xyloside, | 50.8% |
| Wheatoleo) | |

Taking into account the amount of water present in SOPHOCLEAN and Applyclean 6505, the percentage of water present in the composition is 29.54%.

### Composition 2.11 and 2.12: Formulation of a Ready-to-Use Household Degreasing Agent

| | Composition |
|---|---|
| | 2.11 |
| SOPHOCLEAN (DM = 54.5%) | 0.9% |
| Serdet DSK 40 (40% sodium 2-ethylhexyl sulfonate, | 3.3% |
| Elementis) | |
| Appyclean 6781 (60%, C8/C10 Wheat bran, | 0.8% |
| polyglycosides, Wheatoleo) | |
| Appyclean 6552 (60%, amyl xylosides & C10/C12 | 0.8% |
| xylosides, Wheatoleo) | |
| Rhodiasolv RPDE (Rhodia) | 1.0% |
| Sodium metasilicate (30%) | 0.1% |
| Bamboo perfumes | 0.2% |
| Water | quantity |
| | sufficient |
| | for 100% |

### Comparative Example

| | Composition | Comparative |
|---|---|---|
| | 2.12 | composition 3 |
| SOPHOCLEAN (DM = 54.5%) | 0.9% | 0.9% |
| Serdet DSK 40 (40% sodium 2- | 1.3% | 1.3% |
| ethylhexyl sulfonate, Elementis) | | |
| Rhodiasolv RPDE (Rhodia) | 1.0% | 0% |
| Sodium metasilicate (30%) | Quantity | Quantity |
| | sufficient | sufficient |
| | for pH = 6 | pH = 6 |
| Bamboo perfumes | 0.2% | 0.2% |
| Water | Quantity | Quantity |
| | sufficient | sufficient |
| | for 100% | for 100% |

### 3. Demonstration of Reinforced Actions

### 3.1 Dynamic Surface Tension

The dynamic reduction of surface tensions or dynamic surface tension is described in particular by Milton J. Rosen in “Surfactants and interfacial phenomena”, third edition, Wiley-Interscience.

The dynamic surface tensions are an important consideration in numerous industrial fields, and in particular in phytosanitary treatments. The wetting of plants on hydrophobic epicuticular surfaces is encouraged by the reduced surface tension from the first milliseconds of forming the interface. The dynamic surface tensions are measured by means of a tensiometer at maximum bubble pressure, measuring the pressure and the flow of air bubbles exiting a hydrophobic glass capillary.

The relationship between the maximum pressure Pmax, the hydrostatic pressure in the capillary P0, the inner diameter of the tube r and the surface tension γ is expressed according to equation 2.

[equation as in original text]

$$γ = \frac{( {P}_{max} - {P}_{0} ) . r}{2} \qquad eq .2$$

Here a KRUSS device is used of type BP2 and the actions are performed at 25° C.

The actions are performed on sophorolipids at 1 and 0.1% DM at pH=6 as well as on solutions of the composition 2.4 diluted 46 times and 460 times (corresponding to 1% lipid sophoroses and 0.15% Dertol 90 on the one hand and 0.1% sophorolipids and 0.015% Dertol 90 on the other hand).

| | | Surface tension |
|---|---|---|
| | Surface tension | (mN/m) for |
| | (mN/m) for 1% | composition 2.4 |
| Age of interface | sophorolipids at | diluted 46 times |
| (ms) | pH = 6 | (pH = 6) |
| 10 | 48.9 | 42.2 |
| 100 | 42.1 | 38.7 |
| 500 | 39.2 | 26.5 |
| 1000 | 38.3 | 35.7 |
| 10 | 66.3 | 62.6 |
| 100 | 54 | 51.1 |
| 500 | 44 | 42.9 |
| 1000 | 41.3 | 40.4 |

The results show the remarkable effect of the compositions according to the invention on the reduction of surface tension as a function of the duration of creation of the air/liquid interface.

### 3.2 Cleaning Hard Surfaces.

The selected method is the IKW method (IKW—German industrial association for cosmetics, toiletries and detergents) for assessing the performance of cleaning agents for all uses and available at the address: http://www.ikw.org/pdf/broschueren/EQ Allzweck englisch.pdf. The tests are performed using a SHEEN wet abraser of type 903 P&G. White ceramic floor tiles with dimensions 25×40 cm are made dirty by depositing 0.6 to 0.9 g dirt on a strip 8 cm wide by means of an airbrush.

After ageing 24 h in a heat chamber ventilated at 100° C. and 24 h at ambient temperature, the plates are cleaned by means of the abraser and the reference solution recommended by IKW. 5 ml solution is used to impregnate cleaning cloths (a “household” brand) arranged on specific supports of the device. The abraser performs back and forth movements on the ceramic plate at a speed of 20 back and forth movements per minute. The reference solution is used to calibrate the number of back and forth movements and grade the cleaning effect from 0 for an unclean plate to 10 for a perfectly clean plate. The tests performed thereafter with the solutions to be tested are performed at a number of back and forth movements corresponding to grade 2 for the IKW reference solution.

The dirt has the following composition expressed in % weight/weight:

| | |
|---|---|
| Groundnut oil (food quality, supermarket brand) | 74.8% |
| Kaolin monohydrate (Acros Organics) | 23% |
| Carbon black (Acetylene, 50%, Alfa Aesar) | 2% |
| Cross-linking agent (cobalt, cobalt naphthenate, 6%, | 0.2% |
| Aldrich): | |

### Example 3.1: Results of the Cleaning Test on Hard Surfaces

| | Average grade | |
|---|---|---|
| | over a number | |
| | of trials | Standard |
| | >5 (0 to 10) | deviation |
| IKW reference solution | 2 | |
| SOPHOCLEAN 5% DM in (1) | 6.2 | 0.8 |
| 10.4% of the composition 2.1 in (1), | 7 | 1.4 |
| corresponding to 5% SOPHOCLEAN + | | |
| 0.75% methyl rape ester | | |
| 12.5% of the composition 2.7 in (1) | 8.1 | 1.9 |
| corresponding to 5% SOPHOCLEAN + | | |
| 2.5% Rhodiasolv RPDE | | |
| Comparative example 4 (C12/14 | 4 | 0.7 |
| polyglucoside, GLUCOPON 600 of BASF, | | |
| 5% DM in (1)) | | |

(1) is a solution of Na₂HP0₄ of 2.5%, buffering the pH at 7.5.

The results show the effectiveness of SOPHOCLEAN in relation to the comparative example 4, and the remarkable effect of the compositions according to the invention on the result of cleaning hard surfaces.

### Example 3.2: Results of the Cleaning Tests on Hard Surfaces

| | Average grade |
|---|---|
| | after 40 back |
| | and forth |
| | movements |
| Composition 2.12 (with solvent) | 6 |
| Comparative composition 3 (without solvent) | 3.5 |

These results demonstrate the remarkable effect of the compositions according to the invention on the cleaning of hard surfaces.

The invention also relates to the use of at least one sophorolipid as a solubilising agent, in which preferably the form (1) represents 5 to 65%, preferably 10 to 50% of the total mass of sophorolipids.

### 3.3 Effectiveness of Preparations for Combating Weeds According to the Invention.

The preparation of example 2.4 containing terpenic alcohols solubilised by sophorolipids is used as an extemporaneous adjuvant of glyphosate. Glyphosate is a non-selective herbicide, the definition and use of which are described in particular in “The biochemistry and uses of pesticides, 2^{nd} edition” by Kenneth A. Hassall published by VCH (1990). An extemporaneous adjuvant is a preparation added to the phytosanitary preparation at the moment of application and is used to reinforce the action of the latter. An extemporaneous adjuvant or “tank-mix” itself does not contain biologically active molecules.

The glyphosate used is a solution of glyphosate in the form of isopropylamine salt titrated to 620 g/l equivalent glyphosate acid.

The effectiveness of the preparation according to the invention is assessed by forming curves of biological effectiveness, that is by determining the ability of the preparations to reduce the biomass of a target plant as a function of the concentration of herbicide. This method of evaluation is described in particular in “Herbicide Bioassays” by Jens C. Streibig and Per Kudsk, published by CRC press (1993).

The evolution of the biomass with the concentration of herbicide follows the following equation:

Y=C+[(D−C)/(1+exp(−2(a+b log(R·X)))]

where Y is the dry biomass of the plant (plant cut at the base and dried 24 h at 110° C. in a ventilated heat chamber), X is the concentration of herbicide expressed in grams of glyphosate acid per hectare. C, D, a and b being factors determined by analysing the curve according to the least squares method. R represents the factor of effectiveness, and is equal to 1 for the herbicide treatment without adjuvant, and is >1 if the adjuvant reinforces the effectiveness of the herbicide and is <1 in the case of a loss of effectiveness. Its value is determined by applying the above equation with the parameters D, C, a and b of the curve of the herbicide without adjuvant (R=1) according to the least squares method.

The plant tested here is Avena Fatua in pots with a diameter of 10 cm (4 plants per pot, and 4 repetitions). The treatment is performed 3 weeks after the appearance of seedlings at the stage of 2 leaves, by means of a sprayer provided with a nozzle brush making it possible to apply at 2.4 bar 200 L per hectare. The plants are left in a phytotronic chamber making it possible to set the following climatic conditions:
temperature of 23° C./18° C. day/night
70% relative humidity
photoperiod of 16 h (night from 1 h to 9 h in the morning)
light intensity of 158 μmol·m⁻²·s⁻¹

The plants are cut and dried 3 weeks after the treatment.

The adjuvant according to the composition of the invention is added to the herbicide just before the treatment at the dose of 400 g/ha regardless of the concentration of herbicide (varying from 50 to 650 g equivalent acid per hectare).

The result is expressed according to the value of R according to the preceding equation.

| | | Glyphosate iso- |
|---|---|---|
| | | propylamine salt) + |
| | | composition of |
| | Glyphosate (iso- | example 2.4 at |
| | propylamine salt) | 400 L/ha |
| R | 1 | 4.66 |

The statistical analysis according to Anova (p<0.05) shows that the difference between the two values of R is highly significant, and demonstrates the increase in effectiveness obtained by means of the composition 2, 4 according to the invention.

What is claimed is:

1. Cosmetic composition comprising at least one substance that is not water-soluble and is solubilized by a sophorolipid agent and at least water, wherein the sophorolipid agent represents 0.1 to 90% of the mass of the composition, the substance that is not water-soluble represents 0.01 to 65% of the mass of the composition and water 1 to 99.89% of the mass of the composition.

2. The cosmetic composition of claim 1, wherein the sophorolipids have the following general structures 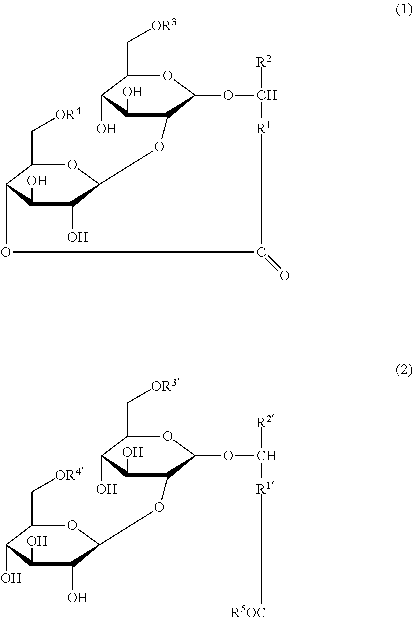
wherein
R¹ and R¹′ are independently of one another saturated hydrocarbon chains or have one or more non-hydroxylated unsaturations or have one or more hydroxyl, linear or ramified groups with 1 to 21 carbon atoms,
R² and R²′ are independently of one another a hydrogen atom or a saturated alkyl radical or have one or more non-hydroxylated unsaturations or have one or more hydroxyl, linear or ramified groups with 1 to 9 carbon atoms,
R³, R⁴, R³′ and R⁴′ are independently of one another a hydrogen atom or an acetyl group, and
R⁵ is in OCH₃ or OH or O⁻M⁺ group, where M⁺ is a metal ion or an organic cation, in particular ammonium salts such as dimethylammonium, trimethylammonium, isopropylammonium, monoethanolammonium, biethanolammonium, triethanolammonium, diphosphonium or O(CH₂)ₘCH₃, with m being between 1 and 11.

3. The cosmetic composition of claim 2, wherein the form (1), represents 5 to 65% of the total mass of sophorolipids.

4. The cosmetic composition of claim 1, wherein the sophorolipid is produced by a method of fermentation and is used in raw form or as an extract from the fermentation process.

5. The cosmetic composition of claim 1, wherein the substance that is not water-soluble is a perfume or an essential oil.

6. The cosmetic composition of claim 1, wherein the substance that is not water-soluble is an active cosmetic.

7. The cosmetic composition of claim 1, wherein the cosmetic composition is a skin or care composition.

8. Method for solubilizing a substance that is not water-soluble, comprising the steps of
(i) adding the substance that is not water-soluble to a concentrated aqueous solution containing 100 to 10% by weight of solubilizing agent;
(ii) agitating at a temperature between 10 and 100° C. for 0.05 minutes to 300 minutes to obtain a pre-mixture;
(iii) adding other constituents to the pre-mixture during continuous agitation; and
(iv) adding the amount of water necessary or the remaining portion of water to obtain a clear solution;
wherein the solubilizing agent consists at least essentially of one or more sophorolipid.

9. The method of claim 8, wherein no other surfactants or solvents are added.

10. The method of claim 8, wherein the sophorolipid is produced by a method of fermentation and is used in raw form or as an extract from the fermentation process.

11. The method of claim 8, wherein the sophorolipids have the following general structures 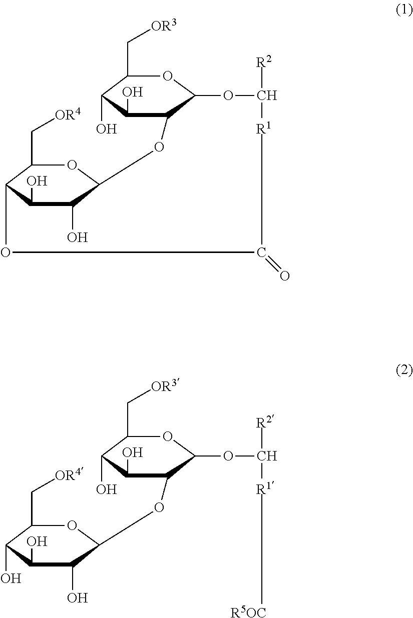
wherein
R¹ and R¹′ are independently of one another saturated hydrocarbon chains or have one or more non-hydroxylated unsaturations or have one or more hydroxyl, linear or ramified groups with 1 to 21 carbon atoms,
R² and R²′ are independently of one another a hydrogen atom or a saturated alkyl radical or have one or more non-hydroxylated unsaturations or have one or more hydroxyl, linear or ramified groups with 1 to 9 carbon atoms,
R³, R⁴, R³′ and R⁴′ are independently of one another a hydrogen atom or an acetyl group, and
R⁵ is in OCH₃ or OH or O⁻M⁺ group, where M⁺ is a metal ion or an organic cation, in particular ammonium salts such as dimethylammonium, trimethylammonium, isopropylammonium, monoethanolammonium, biethanolammonium, triethanolammonium, diphosphonium or O(cH₂)ₘCH₃, with m being between 1 and 11.

12. The method of claim 11, wherein the form (1) represents 5 to 65% of the total mass of sophorolipids.

13. The method of claim 8, wherein the substance that is not water-soluble is a perfume or an essential oil.

14. The method of claim 8, wherein the substance that is not water-soluble is an active cosmetic.
